(19) United States
(12) Patent Application Publication
(10) Pub. No.: US 2023/0343462 A1
(43) Pub. Date: Oct. 26, 2023

(54) MEDICAL INFORMATION PROCESSING SYSTEM, MEDICAL INFORMATION PROCESSING METHOD, AND STORAGE MEDIUM

(71) Applicant: CANON MEDICAL SYSTEMS CORPORATION, Otawara-shi (JP)

(72) Inventor: Yasuko FUJISAWA, Nasushiobara (JP)

(73) Assignee: CANON MEDICAL SYSTEMS CORPORATION, Otawara-shi (JP)

(21) Appl. No.: 18/302,107

(22) Filed: Apr. 18, 2023

(30) Foreign Application Priority Data
Apr. 22, 2022 (JP) 2022-070945

(51) Int. Cl. G16H 50/30; G16H 70/20; G16H 50/20; G16H 10/60

(52) U.S. Cl. CPC G16H 50/30; G16H 70/20; G16H 50/20; G16H 10/60

(57) ABSTRACT

A medical information processing system includes processing circuitry. The processing circuitry is configured to acquire disease state information regarding a disease state presented by a patient, map the disease state information onto a disease ontology to convert the disease ontology into a modified disease ontology in which the state of the patient has been reflected, receive a designation regarding a primary candidate disease, identify a secondary candidate disease different from the primary candidate disease on the basis of the modified disease ontology, and generate first order information regarding the secondary candidate disease.

### CROSS-REFERENCE TO RELATED APPLICATION

The present application claims priority based on Japanese Patent Application No. 2022-070945 filed Apr. 22, 2022, the content of which is incorporated herein by reference.

### FIELD

Embodiments disclosed in the specification and drawings relate to a medical information processing system, a medical information processing method, and a storage medium.

### BACKGROUND

In recent years, a technique (disease ontology) for automatically estimating a specific disease that is an examination target by defining a disease as a total chain of causes and effects of abnormal conditions and analyzing medical data such as medical image data and vital data has become known. In diagnosis using ordinary medical image data, if a certain disease is suspected, medical images are captured in order to perform continuous observation for the certain disease and are analyzed to detect the disease, and the progression of the disease is determined. Further, a technique for presenting diagnostic results with respect to diseases other than a chief complaint disease by using incidental information (patient information and the like) of medical data acquired for diagnosing a disease that is an examination target has also been proposed.

In normal medical inquiry, and the like, a doctor assumes a disease based on a chief complaint. However, in assuming a disease, a disease related to a chief complaint is assumed preferentially, and thus early discovery of, for example, diseases other than the disease related to the chief complaint, for example, other serious diseases in an organ that is a target of the chief complaint, and opportunities of treatment may be missed. In addition, information about complications and side effects of treatment may not be fully recognized during treatment.

### BRIEF DESCRIPTION OF THE DRAWINGS

FIG. 1 is a block diagram showing an example of a configuration of an intra-hospital system 1 of an embodiment.

FIG. 2 is a block diagram showing an example of a configuration of a medical information processing system 100 of an embodiment.

FIG. 3 is a diagram showing an example of the content of a disease ontology.

FIG. 4 is a flowchart showing an example of processing in the medical information processing system 100 of the embodiment.

FIG. 5 is a diagram showing an example of the content of a modified disease ontology in which a primary candidate disease has been reflected.

FIG. 6 is a diagram showing an example of the content of a modified disease ontology in which a secondary candidate disease is identified.

FIG. 7 is a diagram showing an example of the content of a modified disease ontology in which an image diagnostic examination is associated with each element of “disease” and “complications/side effects.”

FIG. 8 is a diagram showing an example of the content of a modified disease ontology showing each element of “disease” and “complications/side effects” for which an imaging examination has been generated.

### DETAILED DESCRIPTION

Hereinafter, a medical information processing system, a medical information processing method, and a storage medium according to embodiments will be described with reference to the drawings. In embodiments, diseases refer to specific diseases such as diabetes, liver fibrosis, cirrhosis, cancer, myocardial infarction, and stroke. Diseases may include pre-diseases that have not yet developed but are not healthy conditions in addition to diseases that have already developed.

A medical information processing system includes processing circuitry. The processing circuitry is configured to acquire disease state information regarding a disease state presented by a patient, map the disease state information onto a disease ontology to convert the disease ontology into a modified disease ontology in which the state of the patient has been reflected, receive a designation regarding a primary candidate disease, identify a secondary candidate disease different from the primary candidate disease on the basis of the modified disease ontology, and generate first order information regarding the secondary candidate disease.

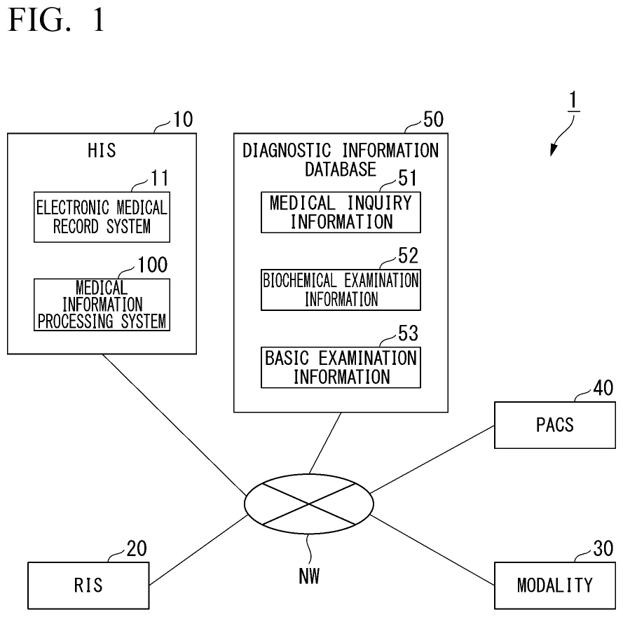

FIG. 1 is a block diagram showing an example of a configuration of an intra-hospital system 1 of an embodiment. The intra-hospital system 1 of the embodiment includes, for example, a hospital information system (hereinafter, HIS) 10, a radiology information system (hereinafter, RIS) 20, a medical image diagnostic apparatus (modality) 30, a picture archiving and communication system (PACS) 40, and a diagnostic information database (hereinafter, DB) 50. The HIS 10 includes an electronic medical record system 11 and a medical information processing system 100. The intra-hospital system 1 is installed in, for example, a medical institution such as a hospital.

The HIS 10, RIS 20, modality 30, PACS 40, and diagnostic information DB 50 are connected via a network NW such that they can communicate. The network NW indicates general information communication networks using telecommunication technology. The network NW includes a telephone communication network, an optical fiber communication network, a cable communication network, a satellite communication network, and the like in addition to a wireless/wired local area network (LAN) such as a hospital backbone LAN and the Internet network.

The HIS 10 is a computer system that supports work within a hospital. Specifically, the HIS 10 has various subsystems including the electronic medical record system 11 and the medical information processing system 100. The various subsystems include, for example, a medical accounting system, a medical appointment system, a hospital visit reception system, and an admission/discharge management system.

The HIS 10 is, for example, a computer such as a server device or a client terminal including a processor such as a central processing unit (CPU), memories such as a read only memory (ROM) and a random access memory (RAM), a display, an input interface, and a communication interface.

A health care professional such as a doctor (hereinafter, a doctor or the like) inputs or refers to various types of information about a patient (hereinafter, patient information) using the electronic medical record system 11 in the HIS 10. Patient information of each patient is managed, for example, by being associated with a patient ID by which each patient can be identified.

The electronic medical record system 11 stores electronic medical records of a plurality of patients. Electronic medical records contain various types of information about patients including patient information. Patient information includes information indicating characteristics of a patient. As characteristics of a patient, for example, the age, sex, physique (height, weight, etc.), suspected diseases, past history, and the like of the patient are conceivable.

A doctor or the like inputs an examination order to the medical information processing system 100 in the HIS 10. The HIS 10 forwards order information including an image examination order to other systems such as the RIS 20. The image examination order is an order that directs image diagnostic analysis. The image examination order may be an order including image diagnostic analysis and an instruction for capturing a medical image that is a target for image diagnostic analysis.

The medical information processing system 100 is a system that transmits instructions (orders) such as examinations and prescriptions to each department in charge. Order information includes physiological examination orders, specimen examination orders, prescription drug orders, dietary maintenance orders, and the like in addition to image examination orders. The medical information processing system 100 serves as an ordering system.

When a doctor or the like inputs an examination order, the HIS 10 causes the medical information processing system 100 to start generation of order information. Before the medical information processing system 100 generates order information, the HIS 10 causes the electronic medical record system 11 to transmit stored patient information of a patient that is an examination target to the medical information processing system 100.

The medical information processing system 100 generates order information on the basis of the examination order input by the doctor or the like, the patient information transmitted by the electronic medical record system 11, diagnostic information transmitted by the diagnostic information DB 50, and other information about the examination target. The medical information processing system 100 transmits the generated order information to the RIS 20 along with some or all of the patient information.

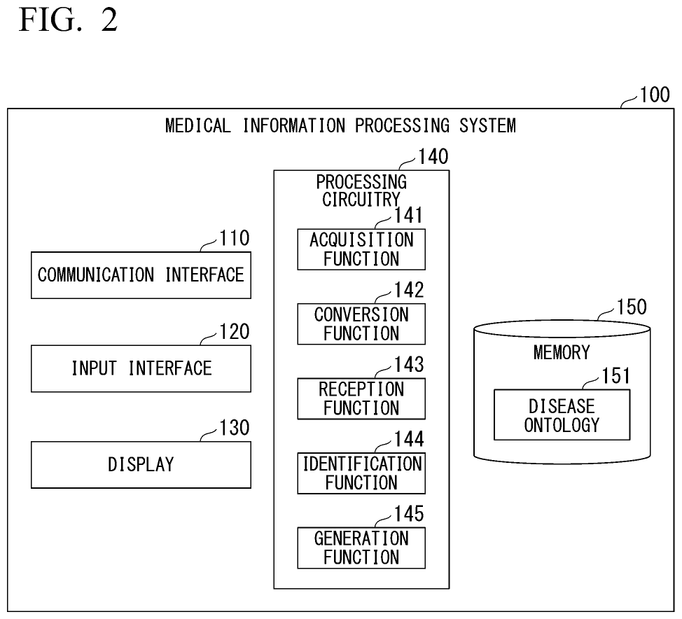

FIG. 2 is a block diagram showing an example of a configuration of the medical information processing system 100 of the embodiment. The medical information processing system 100 includes, for example, a communication interface 110, an input interface 120, a display 130, processing circuitry 140, and a memory 150. Although the communication interface 110, the input interface 120, and the display 130 in the medical information processing system 100 are provided separately from the communication interface, the input interface, and the display included in the HIS 10 and the electronic medical record system 11, they may be shared. The memory 150 is an example of a storage.

The communication interface 110 communicates with external devices such as the RIS 20, the modality 30, and the PACS 40, for example, via a network NW such as a LAN. The communication interface 110 includes, for example, a communication interface such as a network interface card (NIC). The network NW may include the Internet, a cellular network, a Wi-Fi network, a wide area network (WAN), and the like instead of or in addition to the LAN.

The input interface 120 receives various input operations from a medical practitioner or the like, converts the received input operations into electrical signals, and transmits the electrical signals to the processing circuitry 140. When the input operations are performed by a medical practitioner or the like, for example, the input interface 120 generates information according to the input operations. The input interface 120 transmits the generated information according to the input operations to the processing circuitry 140.

The input interface 120 includes, for example, a mouse, a keyboard, a trackball, a switch, a button, a joystick, a touch panel, and the like. The input interface 120 may be, for example, a user interface that receives audio input, such as a microphone. When the input interface 120 is a touch panel, the input interface 120 may also have the display function of the display 130.

The input interface in this specification is not limited to one having physical operation parts such as a mouse and a keyboard. For example, examples of the input interface also include electrical signal processing circuitry that receives an electrical signal corresponding to an input operation from an external input device provided separately from the apparatus and outputs the electrical signal to a control circuit.

A doctor (a doctor in charge) performs diagnosis on a patient and obtains findings related to diseases presented by the patient on the basis of results of medical inquiries, biochemical examination, basic examination, and the like. The doctor inputs diseases indicated by the obtained findings through the input interface 120. The input interface 120 transmits the input disease information regarding the findings of the doctor in charge to the processing circuitry 140. The findings of the doctor in charge include diseases related to the chief complaint (hereinafter, primary candidate diseases).

The display 130 displays various types of information. For example, the display 130 displays an image generated by the processing circuitry 140, a graphical user interface (GUI) for receiving various input operations from an operator, and the like. For example, the display 130 is a liquid crystal display (LCD), a cathode ray tube (CRT) display, an organic electroluminescence (EL) display, or the like.

The processing circuitry 140 includes, for example, an acquisition function 141, a conversion function 142, a reception function 143, an identification function 144, and a generation function 145. The processing circuitry 140 realizes these functions by a hardware processor (computer) executing a program stored in the memory (storage circuit) 150, for example.

The hardware processor is, for example, a circuit (circuitry) such as a CPU, a graphics processing unit (GPU), an application specific integrated circuit (ASIC), or a programmable logic device (e.g., a simple programmable logic device (SPLD), a complex programmable logic device (CPLD), or a field programmable gate array (FPGA)).

Instead of storing the program in the memory 150, the program may be directly incorporated into the circuit of the hardware processor. In this case, the hardware processor realizes the function thereof by reading and executing the program incorporated in the circuit. The aforementioned program may be stored in the memory 150 in advance, or may be stored in a non-transitory storage medium such as a DVD or CD-ROM and may be installed in the memory 150 from the non-transitory storage medium when the non-transitory storage medium is set in a drive device (not shown) of the medical information processing system 100. Further, the program may be stored online (for example, cloud) and the online program may be executed via a communication interface.

The hardware processor is not limited to being configured as a single circuit, and may be configured as one hardware processor by combining a plurality of independent circuits to implement each function. Further, a plurality of components may be integrated into one hardware processor to realize each function. Although the hardware processor, the memory, and the like in the medical information processing system 100 are provided separately from the hardware processor, the memory, and the like of the HIS 10, they may be shared.

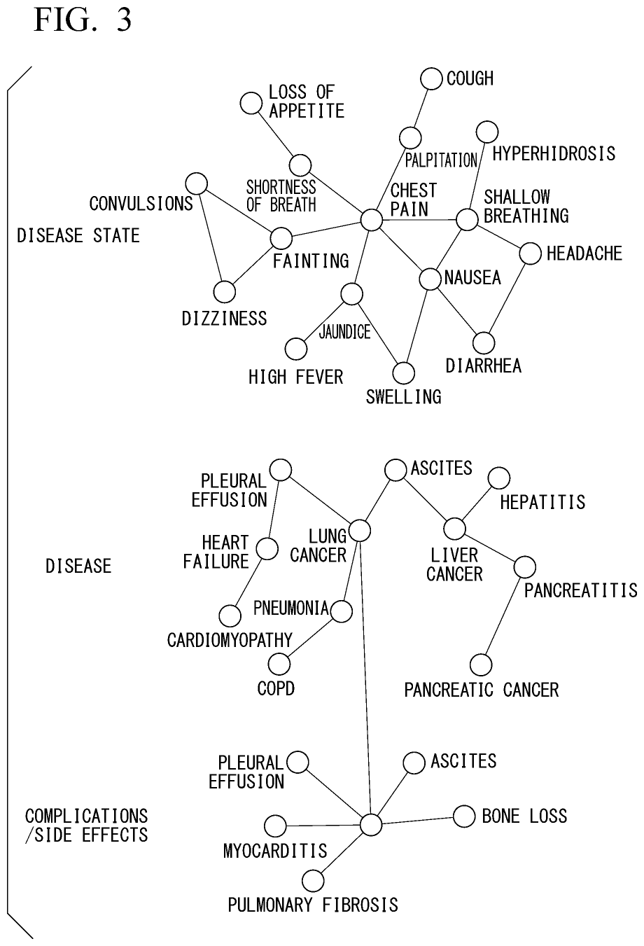

The memory 150 stores a plurality of disease ontologies 151 in which information on diseases is graph-structured. A disease ontology may be stored on the same medium as the program, or may be stored on another medium including online. A disease ontology is read and used, for example, during program execution. FIG. 3 is a diagram showing an example of the content of a disease ontology. The disease ontology 151 is represented, for example, as a disease concept network. In the disease concept network, for example, hierarchies such as “disease state,” “disease,” and “complications/side effects” are presented. Elements are defined in each hierarchy.

For example, in the hierarchy of “disease state,” elements such as “loss of appetite,” “cough,” “shortness of breath,” “palpitation,” “hyperhidrosis,” “convulsions,” “chest pain,” “shallow breathing,” “fainting,” “headache,” “dizziness,” “jaundice,” “nausea,” “high fever,” “swelling,” and “diarrhea” are defined. As elements of the disease state, other elements may be included or some of these elements may not be included. The hierarchy of “disease state” is an example of a layer relating to a disease state.

The “disease” can be, for example, an element classified on the basis of the International Classification of Diseases (ICD-11). The “disease” may be an element structured in application software such as a disease compass. In the hierarchy of “disease,” elements such as “pleural effusion,” “ascites,” “hepatitis,” “heart failure,” “lung cancer,” “liver cancer,” “cardiomyopathy,” “pneumonia,” “pancreatitis,” “COPD,” and “pancreatic cancer” are defined. As elements of the disease, other elements may be included or some of these elements may not be included. The hierarchy of “disease” is an example of a layer related to diseases.

The hierarchy of “complications/side effects” can be defined as a group of diseases that are attributes of a target disease, which are symptoms appearing as complications or side effects when a certain element of the “disease” (hereinafter, target disease) is treated. Elements such as “pleural effusion,” “ascites,” “myocarditis,” “bone loss,” and “pulmonary fibrosis” are defined in the hierarchy of “complications/side effects.” The hierarchy of “complications/side effects” is defined for each type of “disease.” The example of FIG. 3 shows elements of “complications/side effect” when the “disease” is “lung cancer.”

Image diagnostic examination is associated to each element of “disease” and “complications/side effects.” The relationship between each element of “disease” and “complications/side effects” and image diagnostic examination will be described later.

The acquisition function 141 acquires patient information transmitted by the electronic medical record system 11. The acquisition function 141 causes diagnostic information of a patient indicated by the transmitted patient information to be transmitted to the diagnostic information DB 50. The acquisition function 141 acquires diagnostic information transmitted by the diagnostic information DB 50. Both the patient information and the diagnostic information are information regarding a disease state presented by the patient (hereinafter, disease state information). Disease state information is information including patient information and diagnostic information.

The conversion function 142 reads a disease ontology 151 stored in the memory 150 and maps the disease state information to the disease ontology 151. The conversion function 142 converts the disease ontology into a modified disease ontology by mapping the disease state information to generate the modified disease ontology. The modified disease ontology reflects the condition of the patient. The procedure for converting a disease ontology into a modified disease ontology will be further described below.

The reception function 143 performs natural language analysis on the disease information transmitted from the input interface 120, and the like and receives the information as a designation regarding a primary candidate disease. The disease information may be transmitted via the network NW from a device other than the input interface 120, for example, a terminal device such as a user terminal exclusively used by a doctor or the like.

The identification function 144 identifies a secondary candidate disease, which is a disease other than diseases related to a chief complaint and is different from the primary candidate disease received by the reception function 143, on the basis of the modified disease ontology generated by converting the disease ontology 151 through the conversion function 142. Identification of a secondary candidate disease will be further described below.

The generation function 145 generates order information regarding the secondary candidate disease. The generation function 145 also generates order information regarding complications and side effects of the primary candidate disease. The generation function 145 transmits the generated order information regarding the secondary candidate disease and order information regarding complications and side effects of the primary candidate disease to the RIS 20.

Each disease shown as a disease element is associated with an examination for identifying the disease or evaluating the degree of the disease, and information on the associated disease and examination is stored as necessary examination information in the memory 150. The generation function 145 reads necessary examination information associated with the secondary candidate disease identified by the identification function 144 from the memory 150 and generates order information regarding the secondary candidate disease. The order information regarding the secondary candidate disease is an example of first order information. Order information regarding complications and side effects of the primary candidate disease is an example of second order information.

The MS 20 is a computer system that supports operations in an image diagnosis department. The RIS 20 performs cooperation of reservation information with examination equipment, management of examination information, and the like in addition to reservation management of image examination orders in cooperation with the HIS 10. The MS 20 includes, for example, a computer such as a server device or a client terminal including a processor such as a CPU, memories such as a ROM and a RAM, a display, an input interface, and a communication interface.

The modality 30 performs image capturing (imaging) according to imaging conditions (imaging protocol) determined on the basis of, for example, an image examination order. As the modality 30, for example, an X-ray computed tomography apparatus, an X-ray diagnostic apparatus, a magnetic resonance imaging apparatus, an ultrasonic diagnostic apparatus, a nuclear medicine diagnostic apparatus, and the like are conceivable. The modality 30 is operated by an operator such as a doctor (radiologist) or a radiographer. The modality 30 transmits a medical image (image data) generated by imaging to the PACS 40.

The PACS 40 is a computer system that receives medical images transmitted by the modality 30 and stores them in a database. The PACS 40 transmits (forwards) medical images stored in the database in response to a request from a client. The PACS 40 includes a server/computer including a processor such as CPU, memories such as a ROM and a RAM, a display, an input interface, and a communication interface.

Information on a patient that is an imaging target and imaging is attached to medical images stored in the PACS 40 as supplementary information. The supplementary information includes information such as a patient ID, an examination ID, imaging conditions (imaging protocol), and the like in a format conforming to Digital Imaging and Communication in Medicine (DICOM) standards, for example. The PACS 40 stores medical images of a plurality of patients captured in the past.

The diagnostic information DB 50 stores information obtained by diagnosing patients (hereinafter, diagnostic information). Diagnostic information stored in the diagnostic information DB 50 includes medical inquiry information 51, biochemical information 52, and basic examination information 53, for example. The medical inquiry information 51 includes, for example, information obtained by a doctor or the like inquiring of a patient for medical examination. The biochemical information 52 includes, for example, information obtained by biochemical examinations. The basic examination information 53 includes, for example, information on basic examinations executed before medical inquiry of a doctor, such as electrocardiogram information.

The diagnostic information DB 50 may be included in the electronic medical record system 11. In this case, when transmitting patient information to the medical information processing system 100, the electronic medical record system 11 may read diagnostic information indicated by the patient information from the diagnostic information DB 50 and transmit the diagnostic information along with the patient information to the medical information processing system 100. Diagnostic information may include results of image diagnostic examinations.

The configuration of the intra-hospital system 1 is not limited to the above-described one. In the intra-hospital system 1, some elements thereof may be integrated. For example, the HIS 10 and the RIS 20 may be integrated into one system.

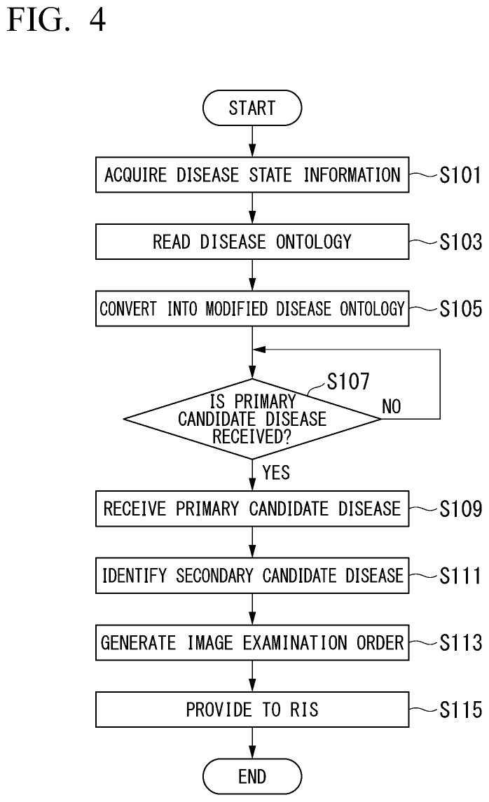

Next, processing in the medical information processing system 100 will be described. FIG. 4 is a flowchart showing an example of processing in the medical information processing system 100 of the embodiment. The medical information processing system 100 first acquires patient information transmitted from the electronic medical record system 11 and diagnostic information transmitted from the diagnostic information DB 50 using the acquisition function 141 to acquire disease state information (step S101).

Subsequently, the conversion function 142 reads out a disease ontology 151 stored in the memory 150 (step S103). Subsequently, the conversion function 142 maps the disease state information to the read disease ontology 151 to convert the disease ontology into a modified disease ontology (step S105).

Subsequently, the reception function 143 determines whether or not disease information (primary candidate disease) transmitted from the input interface 120 has been received (step S107). Upon determining that the disease information has not been received, the reception function 143 repeats processing of step S107. Upon determining that the disease information has been received, the reception function 143 receives a disease based on the received disease information as a primary candidate disease (step S109). Subsequently, the conversion function 142 reflects the primary candidate diseases received by the reception function 143 in the modified disease ontology.

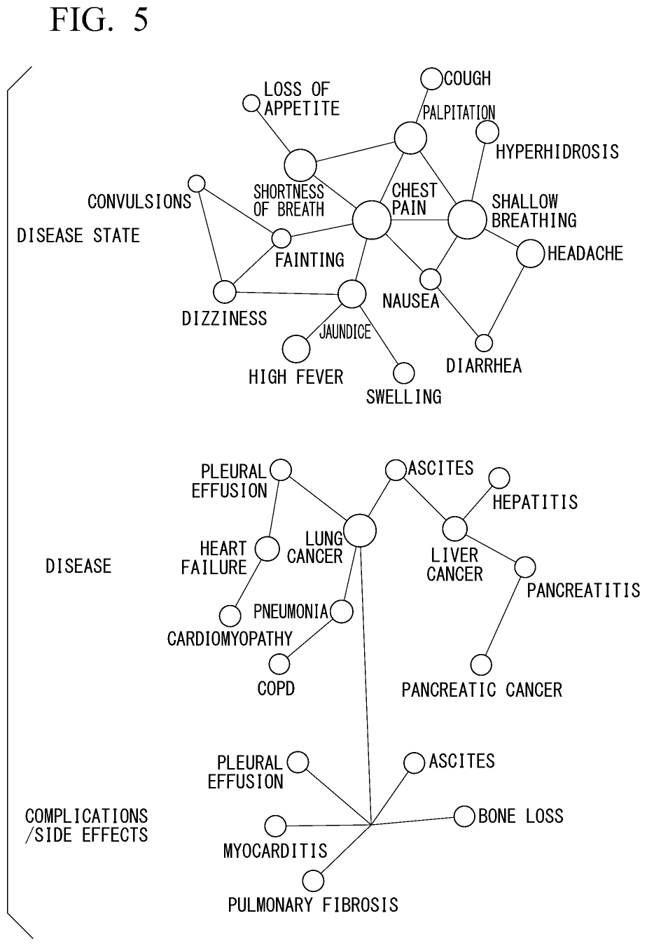

FIG. 5 is a diagram showing an example of the content of a modified disease ontology in which a primary candidate disease has been reflected. At the time of converting the disease ontology into the modified disease ontology, the conversion function 142 graphs each element of “disease state” on the basis of medical inquiry information, biochemical examination information, and basic examination information included in the diagnostic information. For example, the conversion function 142 performs natural language analysis on text information included in the medical inquiry information, and graphs elements of “disease state” included in the medical inquiry information using the number of each element as an index. For example, as the number included in the medical inquiry information increases, each element in “disease state” of the disease ontology is graphed larger.

The graphed elements are indicated, for example, in sizes of marks in FIG. 5. In the example shown in FIG. 5, text information includes many elements such as “chest pain,” “shortness of breath,” and “shallow breathing” and these elements are graphed largely. For this reason, marks indicating elements such as “chest pain,” “shortness of breath,” and “shallow breathing” are displayed largely.

Here, the number of each element included in the medical inquiry information is used as an index to graph elements, but it is also possible to graph elements on the basis of an index other than the number. Element may be graphed on the basis of an index other than the number, for example, the magnitude of influence, and a degree of emphasis, or the like, or elements may be graphed by evaluating each index multi-dimensionally. In this case, representation of a mark may be changed for each index to be graphed, for example, the degree of influence may be indicated by color (density), for example, and the degree of emphasis may be indicated in a shape (circle, square, triangle, or the like).

The conversion function 142 further graphs elements of “disease state” in the disease ontology on the basis of biochemical examination information and basic examination information. For example, if biochemical examinations and basic examinations show results in which a symptom regarding each element is easily viewed, the element is graphed largely. The index of each element included in biochemical examinations and basic examinations may be an index other than the number as in medical inquiry information.

Further, the conversion function 142 graphs elements of “disease” in the disease ontology on the basis of the disease information received by the reception function 143. In the example shown in FIG. 5, the reception function 143 receives “lung cancer” as disease information. In this case, the element of “lung cancer” in the disease is graphed largely. In addition, since “lung cancer” is graphed most largely as a “disease,” elements of “complications/side effects” when the “disease” is “lung cancer” are selected as elements of “complications/side effects.”

Subsequently, the identification function 144 identifies a secondary candidate disease (step S111). The identification function 144 uses the primary candidate disease received by the reception function 143 and the graphed elements in the disease state in the modified disease ontology to identify a secondary candidate disease. The identification function 144 may identify the secondary candidate disease in any manner. The identification function 144 infers a disease with a high morbidity probability and identifies it as a secondary candidate disease, for example, on the basis of the primary candidate disease and each graphed element of the disease state in the modified disease ontology. At the time of inferring a disease with a high morbidity probability, for example, a trained model generated by machine learning may be used or a rule-based model may be used.

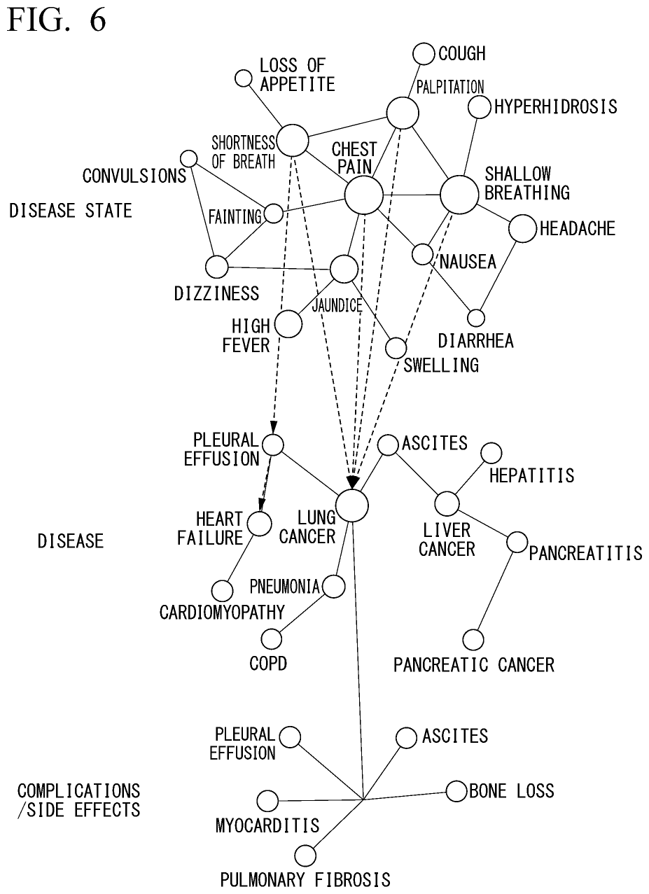

FIG. 6 is a diagram showing an example of the content of a modified disease ontology in which a secondary candidate disease is identified. In the example shown in FIG. 6, a primary candidate disease is “lung cancer” and “chest pain,” “palpitation,” “shortness of breath,” “shallow breathing,” and the like in the disease state are graphed largely. “Heart failure” is identified as a secondary candidate disease from these results.

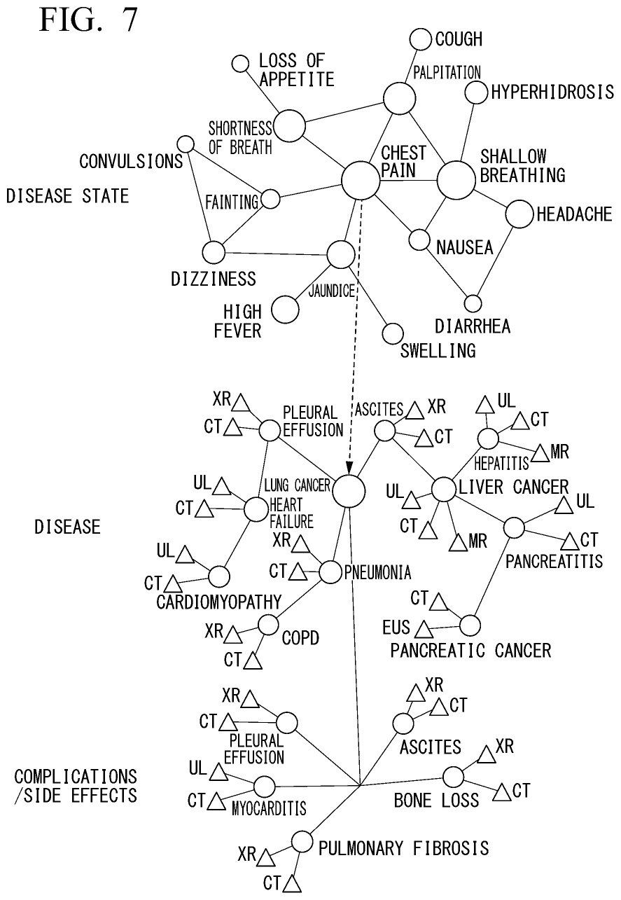

In addition, image diagnostic examinations are associated with each element of “disease” and “complications/side effects.” FIG. 7 is a diagram showing an example of the content of a modified disease ontology in which diagnostic imaging examinations are associated with each element of “disease” and “complications/side effects.” For example, “UL (ultrasonography)” and “CT (Computed Tomography)” are associated with “heart failure” in “disease.” Further, “UL” and “CT” are associated with “myocarditis” in “complications/side effects.” An image diagnostic examination protocol is coded and easily identified.

Subsequently, the generation function 145 generates an image examination order for each element of “complications/side effects” associated with the secondary candidate disease identified by the identification function 144 and the primary candidate disease received by the reception function 143 (step S113). The generation function 145 includes, for example, image examinations required to examine the secondary candidate disease identified by the identification function 144 in the image examination order. The generation function 145 includes, in the image examination order, an image examination required to examine an element with a high need for examination in “complications/side effects” associated with the primary candidate disease.

In selection of an element with a high need for examination from among the elements of “complications/side effects,” the generation function 145 calculates a degree of recommendation of execution of examination for each element and performs weighting depending on the degree of recommendation of execution. In calculation of the degree of recommendation of execution, the generation function 145 uses text information, basic examination information, clinical practice guidelines for primary candidate disease, and past examination results of the patient included in the medical inquiry information.

The generation function 145 calculates a degree of recommendation of execution, Ei, for example, using the following formula (1) using an occurrence probability Ri, a recommendation rank Si, and a change coefficient Ci.

Ei=Ri×Si×Ci (1)

The occurrence probability Ri is a probability of occurrence of a state in which complications or side effects of the primary candidate disease are easily developed. The generation function 145 calculates the occurrence probability Ri, for example, on the basis of patient's symptoms extracted by natural language analysis of the text information included in the medical inquiry information and the basic examination information. The recommendation rank Si is identified on the basis of a recommendation rank defined in clinical practice guidelines. For example, the recommendation rank Si is weighted in 5 stages in which “5” indicates that examination is highly recommended and “1” indicates that examination is not recommended in the clinical practice guidelines. The generation function 145 may use a degree of attention in the clinical practice guideline, a degree of severity at the time of onset, and the like.

The change coefficient Ci is calculated, for example, when the patient has undergone any examination in the past. If the patient has undergone an examination in the past, results of the past examination are included as information in the modified disease ontology. Therefore, the change coefficient Ci is calculated by the following formula (2), for example, using change PCi in patient statement and change BCi in the basic examination information. The change PCi in the patient statement and the change BCi in the basic examination information are set, for example, in 4 stages in which “1” indicates that there is no change or change is in a direction of improvement and “5” indicates larger one according to the magnitude of a degree of deterioration when change is in a direction of deterioration.

Ci=PCi×BCi (2)

The generation function 145 calculates the degree of recommendation of execution using formula (1) and generates an image examination order regarding each element of “complications/side effects” associated with the primary candidate disease using weighting according to the calculated degree of recommendation of execution. For example, the generation function 145 may include, in the image examination order, an image examination of an element for which the degree of recommendation of execution exceeds a predetermined threshold value among the plurality of elements, or include, in the image examination order, image examinations of a predetermined number of elements with high degrees of recommendation of execution.

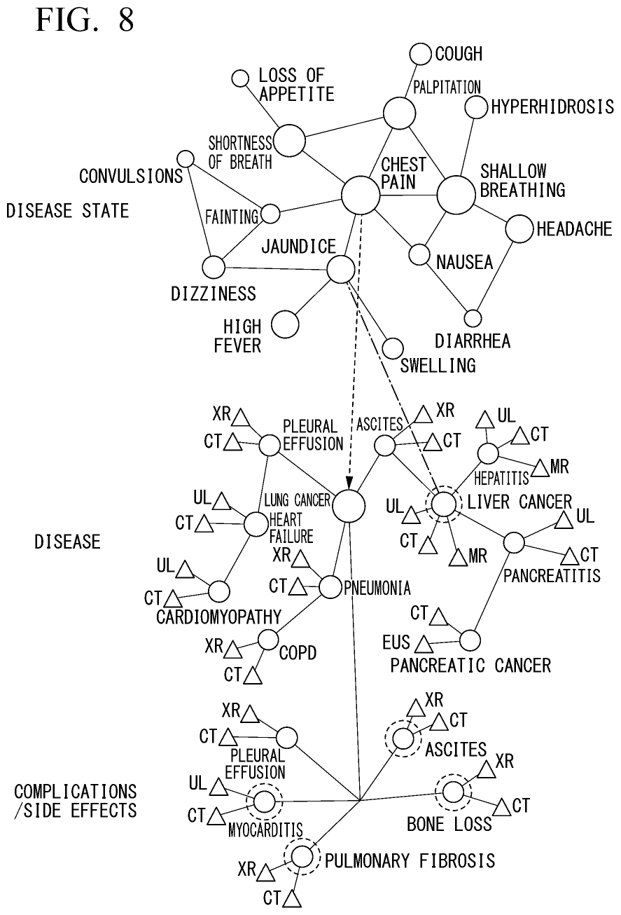

FIG. 8 is a diagram showing an example of the content of a modified disease ontology showing each element of “disease” and “complications/side effects” for which an image examination order has been generated. In the example shown in FIG. 8, image examination orders have been generated for “liver cancer” in “disease” and “ascites,” “myocarditis,” “pulmonary fibrosis,” and “bone loss” in “complications/side effects.”

The generation function 145 may select image examinations to be included in an image examination order on the basis of whether image diagnostic examination and analysis of a disease can be performed in accordance with an image diagnostic examination protocol indicated by a doctor. For example, the generation function 145 may include image examinations that can be performed in accordance with the image diagnostic examination protocol indicated by the doctor in image examinations and exclude image examinations that cannot be performed in accordance with the image diagnostic examination protocol indicated by the doctor from the image examinations. In this case, the generation function 145 may suggest that the doctor will perform an image examination that cannot be performed in accordance with the image diagnostic examination protocol instructed by the doctor as an additional examination.

Subsequently, the generation function 145 transmits the generated image examination order to the RIS 20 (step S115). Accordingly, the medical information processing system 100 ends processing of the flow shown in FIG. 4.

The medical information processing system 100 of the embodiment infers a secondary candidate disease other than a primary candidate disease using a disease ontology and generates order information with respect to the secondary candidate disease. The disease ontology used here is a modified disease ontology in which patient's conditions have been reflected, which is obtained by mapping disease state information onto the disease ontology to convert the disease ontology. Therefore, it is possible to easily discover diseases other than diseases related to a chief complaint.

In addition, the medical information processing system 100 of the embodiment generates examination orders regarding complications and side effects of a primary candidate disease. Therefore, complications and side effects of the primary candidate disease can also be easily discovered. Further, complications and side effects for which examination orders will be generated are determined on the basis of a degree of recommendation of execution. Therefore, it is possible to curb excessive examination for complications and side effects.

Although the medical information processing system 100 is provided in the HIS 10 in the above embodiment, the medical information processing system 100 may be provided in other locations. For example, the medical information processing system 100 may be provided independently of the HIS 10 or may be provided in a user terminal or the electronic medical record system 11 operated by a doctor.

Although the medical information processing system 100 generates order information for an image examination order in the above embodiment, the medical information processing system 100 may generate order information for one other than an image examination order. The medical information processing system 100 may generate order information for, for example, a physiological examination order or a specimen examination order.

According to at least one embodiment described above, it is possible to easily discover a secondary disease other than a primary disease by including an acquirer that acquires disease state information regarding a disease state presented by a patient, a converter that maps the disease state information onto a disease ontology to convert the disease ontology into a modified disease ontology in which the state of the patient has been reflected, a receiver that receives a designation regarding a primary candidate disease, an identifier that identifies a secondary candidate disease different from the primary candidate disease on the basis of the modified disease ontology, and a generator that generates first order information regarding the secondary candidate disease.

The embodiment described above can be represented as follows.

A medical information processing apparatus including processing circuitry,

wherein the processing circuitry is configured to:

acquire disease state information regarding a disease state presented by a patient;

map the disease state information onto a disease ontology to convert the disease ontology into a modified disease ontology in which the state of the patient has been reflected;

receive a designation regarding a primary candidate disease;

identify a secondary candidate disease different from the primary candidate disease on the basis of the modified disease ontology; and

generate first order information regarding the secondary candidate disease.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing system comprising processing circuitry configured to:
acquire disease state information regarding a disease state presented by a patient;
map the disease state information onto a disease ontology to convert the disease ontology into a modified disease ontology in which the state of the patient has been reflected;
receive a designation regarding a primary candidate disease;
identify a secondary candidate disease different from the primary candidate disease on the basis of the modified disease ontology; and
generate first order information regarding the secondary candidate disease.

2. The medical information processing system according to claim 1, wherein the disease ontology includes at least a layer related to disease states and a layer related to diseases.

3. The medical information processing system according to claim 1, further comprising a storage storing the disease ontology.

4. The medical information processing system according to claim 1, wherein the processing circuitry is further configured to generate second order information regarding at least any of complications or side effects associated with the primary candidate disease.

5. The medical information processing system according to claim 4, wherein the processing circuitry is further configured to generate the second order information on the basis of a degree of recommendation of execution for at least one of the complications or the side effects.

6. The medical information processing system according to claim 5, wherein the processing circuitry is further configured to calculate the degree of recommendation of execution on the basis of at least one of a probability of occurrence of at least one of the complications or the side effects, a recommendation rank, or a change coefficient.

7. The medical information processing system according to claim 6, wherein the recommendation rank is determined on the basis of clinical practice guidelines for the primary candidate disease.

8. The medical information processing system according to claim 6, wherein the change coefficient is determined on the basis of past examination results of the patient.

9. A medical information processing method, using a computer, comprising:
acquiring disease state information regarding a disease state presented by a patient;
mapping the disease state information onto a disease ontology to convert the disease ontology into a modified disease ontology in which the state of the patient has been reflected;
receiving a designation regarding a primary candidate disease;
identifying a secondary candidate disease different from the primary candidate disease on the basis of the modified disease ontology; and
generating first order information regarding the secondary candidate disease.

10. A computer-readable non-transitory storage medium storing a program causing a computer to:
acquire disease state information regarding a disease state presented by a patient;
map the disease state information onto a disease ontology to convert the disease ontology into a modified disease ontology in which the state of the patient has been reflected;
receive a designation regarding a primary candidate disease;
identify a secondary candidate disease different from the primary candidate disease on the basis of the modified disease ontology; and
generate first order information regarding the secondary candidate disease.
